(19) United States
(12) Patent Application Publication
(10) Pub. No.: US 2024/0161293 A1
(43) Pub. Date: May 16, 2024

(54) MULTI-LABEL CLASSIFICATION METHOD FOR MEDICAL IMAGE

(71) Applicant: HTC Corporation, Taoyuan City (TW)

(72) Inventors: Zhe-Ting LIAO, Taoyuan City (TW); Yu-Shao PENG, Taoyuan City (TW)

(21) Appl. No.: 18/510,672

(22) Filed: Nov. 16, 2023

Related U.S. Application Data
(60) Provisional application No. 63/383,918, filed on Nov. 16, 2022.

(51) Int. Cl. G06T 7/00; G06T 7/194; G06V 10/764; G06V 20/70; G16H 30/40

(52) U.S. Cl. CPC G06T 7/0012; G06T 7/194; G06V 10/764; G06V 20/70; G16H 30/40; G06T 2207/10081; G06T 2207/20084; G06T 2207/30016

(57) ABSTRACT

A multi-label classification method for generating labels annotated on medical images. An initial dataset including medical images and partial input labels is obtained. The partial input labels annotate a labeled part of abnormal features on the medical images. A first multi-label classification model is trained with the initial dataset. Difficulty levels of the medical images in the initial dataset are estimated based on predictions generated by the first multi-label classification model. The initial dataset is divided based on the difficulty levels of the medical images into different subsets. A second multi-label classification model is trained based on subsets with gradually increasing difficulty levels during different curriculum learning rounds. Predicted labels annotated on the medical images are generated about each of the abnormal features based on the second multi-label classification model.

### RELATED APPLICATIONS

This application claims the priority benefit of U.S. Provisional Application Ser. No. 63/383,918, filed Nov. 16, 2022, which is herein incorporated by reference.

### BACKGROUND

### Field of Invention

The disclosure relates to a classification method for medical images. More particularly, the disclosure relates to a classification method for generating multiple labels annotated on one medical image.

### Description of Related Art

Medical image examinations, such as X-ray scan, Magnetic Resonance Imaging (MRI) and Computed Tomography (CT) scan, are tools to evaluate conditions of patients. To identify abnormal conditions appeared in the medial images requires experienced medical personnel. It is desired to establish a machine-learning model capable of detecting and distinguishing abnormal conditions within the medial images to improve the efficiency and cost effectiveness of the medical image examinations.

### SUMMARY

An embodiment of the disclosure provides a multi-label classification method, which include steps as below. An initial dataset including medical images and partial input labels is obtained. The partial input labels annotate a labeled part of abnormal features on the medical images. A first multi-label classification model is trained with the initial dataset. Difficulty levels of the medical images in the initial dataset are estimated based on predictions generated by the first multi-label classification model. The initial dataset is divided based on the difficulty levels of the medical images into at least a first subset and a second subset. The second subset is estimated to have a higher difficulty level compared to the first subset. A second multi-label classification model is trained with the first subset during a first curriculum learning round. The second multi-label classification model is trained with the first subset and the second subset during a second curriculum learning round. Predicted labels annotated on the medical images are generated about each of the abnormal features based on the second multi-label classification model.

Another embodiment of the disclosure provides a multi-label classification system, which include a storage unit and a processing unit. The storage unit is configured to store computer-executable instructions. The processing unit is coupled with the storage unit. The processing unit is configured to execute the computer-executable instructions to implement a first multi-label classification model and a second multi-label classification model. The processing unit is configured to obtain an initial dataset comprising medical images and partial input labels, the partial input labels annotating a labeled part of abnormal features on the medical images. The processing unit is configured to train the first multi-label classification model with the initial dataset. The processing unit is configured to estimate difficulty levels of the medical images in the initial dataset based on predictions generated by the first multi-label classification model. The processing unit is configured to divide the initial dataset based on the difficulty levels of the medical images into at least a first subset and a second subset. The second subset is estimated to have a higher difficulty level compared to the first subset. The processing unit is configured to train the second multi-label classification model with the first subset during a first curriculum learning round. The processing unit is configured to train the second multi-label classification model with the first subset and the second subset during a second curriculum learning round. The processing unit is configured to utilize the second multi-label classification model to generate predicted labels annotated on the medical images about each of the abnormal features.

It is to be understood that both the foregoing general description and the following detailed description are by examples, and are intended to provide further explanation of the invention as claimed.

### BRIEF DESCRIPTION OF THE DRAWINGS

The disclosure can be more fully understood by reading the following detailed description of the embodiment, with reference made to the accompanying drawings as follows:

FIG. 1 is a flow chart diagram illustrating a multi-label classification method according to some embodiments of the disclosure.

FIG. 2 is a block diagram illustrating a multi-label classification system for performing the multi-label classification method shown in FIG. 1 in some embodiments of the disclosure.

FIG. 3 is a schematic diagram illustrating an image matting step performed on an original medical image to generate a medical image after pre-processing.

FIG. 4 is a schematic diagram illustrating an image windowing step performed on an original medical image to generate medical images after pre-processing.

FIG. 5 is a schematic diagram illustrating an image stacking step performed on a sequence of original medical images to generate a medical image after pre-processing.

FIG. 6 is a schematic diagram illustrating the multi-label classification method in FIG. 1 in some embodiments.

FIG. 7 is a flow chart diagram illustrating further steps of the multi-label classification method in FIG. 1 according to some embodiments of the disclosure.

FIG. 8 is a schematic diagram illustrating information about mismatched predicted labels on the displayer according to some embodiments.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

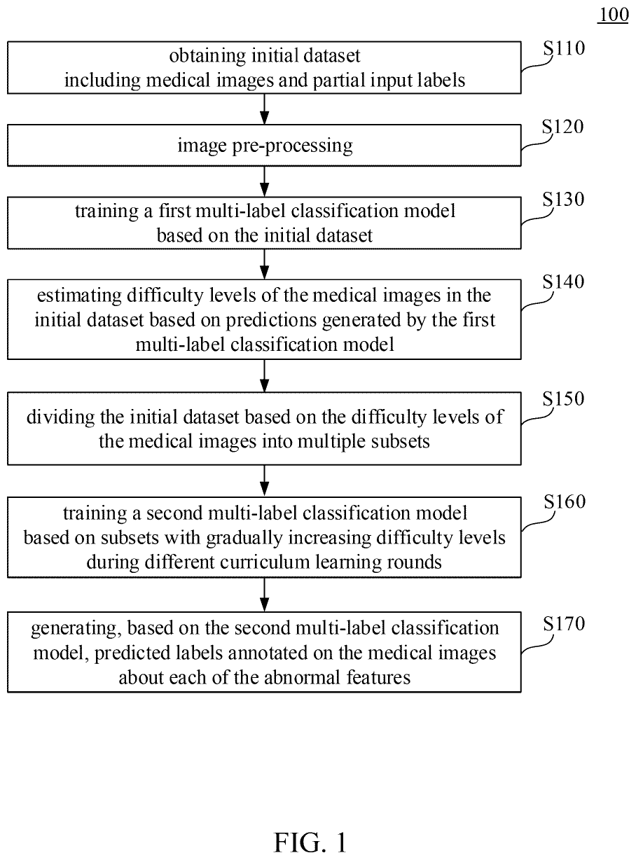

Reference is made to FIG. 1, which is a flow chart diagram illustrating a multi-label classification method 100 according to some embodiments of the disclosure. The multi-label classification method 100 is configured to generate multiple labels reflecting different abnormal features potentially appeared in each one of medical images.

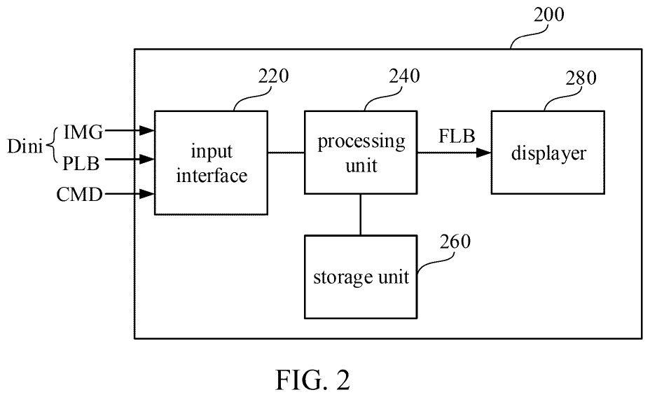

Reference is further made to FIG. 2, which is a block diagram illustrating a multi-label classification system 200 for performing the multi-label classification method 100 shown in FIG. 1 in some embodiments of the disclosure. As shown in FIG. 2, the multi-label classification system 200 includes an input interface 220, a processing unit 240, a storage unit 260 and a displayer 280. In some embodiments, the multi-label classification system 200 can be a computer, a smartphone, a tablet, an image processing server, a data server, a tensor computing server or any equivalent processing device.

The storage unit 260 is configured to store computer-executable instructions. The processing unit 240 is coupled with the input interface 220, the storage unit 260 and displayer 280. The processing unit 240 is configured to execute the computer-executable instructions to implement multi-label classification models discussed in following embodiments.

As shown in FIG. 1 and FIG. 2, step S110 is executed, by the input interface 220 of the multi-label classification system 200, configured to receive/obtain an initial dataset Dini from a data source (not shown in figures). In some embodiments, the data source can be a data server storing medical reports in hospitals. In some embodiments, the initial dataset Dini includes medical images IMG and partial input labels PLB.

The input interface 220 is configured to receive the initial dataset Dini. The input interface 220 can include a data transmission interface, a wireless communication circuit, a keyboard, a mouse, a microphone or any equivalent input device. The processing unit 240 is coupled with the input interface 220, the storage unit 260 and the displayer 280. The storage unit 260 is configured to store a program code. The program code stored in the storage unit 260 is configured for instructing the processing unit 240 to execute the multi-label classification method 100 shown in FIG. 1. In some embodiments, the processing unit 240 can be a processor, a graphic processor, an application specific integrated circuit (ASIC) or any equivalent processing circuit. The displayer 280 can be a display panel, a monitor, a projector, a touch screen or any equivalent displayer.

In some embodiments, the medical images IMG include head computed tomography (CT) images captured from patients with intracranial hemorrhage (ICH). In practices, there are different abnormal features potentially existed in the medical images IMG corresponding to different types of intracranial hemorrhages, such as intraparenchymal hemorrhage (IPH), intraventricular hemorrhage (IVH), subarachnoid hemorrhage (SAH), subdural intracranial hemorrhage (SDH) and epidural hemorrhage (EDH).

Labeling different abnormal features of intracranial hemorrhages on the medical images (e.g., computed tomography scan or magnetic resonance imaging scan) requires careful analysis and understanding of the specific characteristics of each type of intracranial hemorrhage. A brief overview of the five types of intracranial hemorrhage is listed as below in Table 1:

TABLE 1

| Types of ICH | Definition | Characteristics |
|---|---|---|
| Intraparenchymal | Bleeding within the | Appears as a hyperdense |
| Hemorrhage | brain tissue itself. | (bright) region on CT scans |
| (IPH) | | within the brain parenchyma. |
| Intraventricular | Bleeding into the | Blood within the ventricles, |
| Hemorrhage | ventricles of the | often visible as hyperdensity |
| (IVH) | brain. | on CT scans. It can also cause |
| | | ventricular enlargement. |
| Subarachnoid | Bleeding into the | Appears as diffuse or focal |
| Hemorrhage | space between the | hyperdensity on CT scans in |
| (SAH) | arachnoid and pia | the subarachnoid space |
| | mater layers of | |
| | the meninges | |
| | (subarachnoid space) | |
| Subdural | Accumulation of blood | Crescent-shaped or lens- |
| Hematoma | between the dura | shaped hyperdensity on CT |
| (SDH) | mater and the | scans, often along the cerebral |
| | arachnoid membrane. | convexity. |
| Epidural | Accumulation of | Biconvex or lens-shaped |
| Hematoma | blood between the | hyperdensity on CT scans, |
| (EDH) | dura mater and | often associated with skull |
| | the skull. | fractures. |

Accurate labeling of intracranial hemorrhage is pivotal for effective treatment and prognosis. Identifying intracranial hemorrhage types like IPH, IVH, SAH, SDH and EDH is essential for precise medical intervention. Each type necessitates distinct treatment strategies, such as surgery, drainage, or conservative management, tailored to its location and characteristics. Proper diagnosis guides healthcare providers in preventing complications like increased intracranial pressure and cerebral herniation. It also aids in monitoring the patient's progress, ensuring timely adjustments in therapy if needed. In essence, precise intracranial hemorrhage labeling is critical for personalized and effective medical care, significantly impacting patient outcomes. In some embodiments, the multi-label classification method 100 in some embodiments provides an effective way to train a multi-label classification model, which is capable of identifying different types of intracranial hemorrhage and generating corresponding labels.

In some cases, a large amount of labeled data is required to train the multi-label classification model. It takes a lot of time to manually label each type of abnormal features about IPH, IVH, SAH, SDH and EDH on every medical image in a training dataset. In order to speed up labeling, the medical personnel may search for images that match the keywords from the medical reports of the images. For example, if it is desired to retrieve images with EDH, the medical personnel may enter keywords such as “EDH”, “epidural hemorrhage” and retrieve medical images with the keyword in the corresponding medical report. The image will be marked “EDH” directly, or it will go through a further process of confirmation by the medical personnel. This annotation method may have data defects.

Firstly, some labels will be missing in the annotated data. For example, one ICH medical image may have a label about one abnormal feature of EDH, and this ICH medical image may actually have other abnormal features (e.g., IPH, IVH, SAH or SDH). If these other abnormal features are not recorded in the medical report. Labels automatically generated from the medical report will not include full annotations on this medical image. Secondly, the labels automatically generated from the medical report may be misinterpreted. For example, if the medical report says “result without EDH” or “the patient has no EDH”, a correct label corresponding to the EDH features shall be “negative”. In some cases, an automatically generated label may capture the keyword “EDH” without interpreting context and labeled as “positive” by mistake.

In some embodiments, the partial input labels PLB in the initial dataset Dini can be automatically generated (e.g., matching the keywords from the medical reports) from the medical reports. The partial input labels PLB annotate a labeled part of abnormal features on the medical images IMG. Reference is further made to Table 2, which is a list of partial input labels PLB annotated on medical images IMG1-IMGk in the initial dataset Dini according to a demonstrational example.

TABLE 2

| | IPH | IVH | SAH | SDH | EDH |
|---|---|---|---|---|---|
| IMG1 | positive | x | negative | x | x |
| IMG2 | x | positive | x | negative | x |
| IMG3 | negative | x | positive | x | x |
| IMG4 | x | x | x | positive | negative |
| . . . | . . . | . . . | . . . | . . . | . . . |
| IMGk | x | negative | x | x | positive |

As shown in Table 2, regarding to the first medical image IMG1, the partial input labels PLB includes a “positive” input label for the first abnormal feature IPH on the medical images IMG1, and a “negative” input label for the third abnormal feature SAH on the first medical images IMG1. The partial input labels PLB indicate that the first abnormal feature IPH existed in the first medical image IMG1 and the third abnormal feature SAH does not existed in the first medical image IMG1. It is noticed that, there is an unlabeled part of the abnormal features (for IVH, SDH and EDH) in the partial input labels PLB about the first medical image IMG1. The unlabeled part of the abnormal features is unknown in the partial input labels PLB corresponding to the first medical image IMG1. In other words, whether the first medical image IMG1 has the abnormal features IVH, SDH and EDH are uncertain according to the initial dataset Dini. In this case, the partial input labels PLB include 2 confirmed labels relative to the medical image IMG1.

Similarly, as shown in Table 2, regarding to the second medical image IMG2, the partial input labels PLB annotate “positive” for the second abnormal feature IVH on the second medical images IMG2, and “negative” for the fourth abnormal feature SDH on the second medical images IMG2. There is an unlabeled part of the abnormal features (for IPH, SAH and EDH) in the partial input labels PLB about the second medical image IMG2. Whether the second medical image IMG2 has the abnormal features IPH, SAH and EDH are currently uncertain according to the initial dataset Dini.

In other words, each of the medical images IMG1-IMGk is potentially subject to M abnormal features (in this demonstrational example, M=5). The partial input labels indicate positive or negative predictions about N abnormal features in one medical image. M and N are positive integers and M>N.

In some embodiments, as shown in FIG. 1 and FIG. 2, step S120 is executed by the processing unit 240 to perform image pre-processing to the medical images IMG in the initial dataset Dini. In some embodiments, the image pre-processing includes at least one of image matting, image windowing and sequential image stacking.

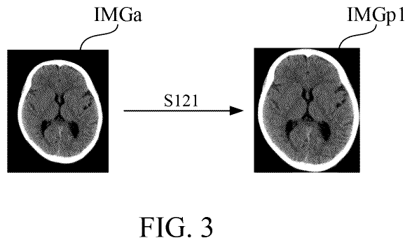

Reference is further made to FIG. 3, which is a schematic diagram illustrating an image matting step S121 performed on an original medical image IMGa to generate a medical image IMGp1 after pre-processing. As shown in FIG. 3, during the image matting step S121, a brain region is cropped out from a black background in the original medical image IMGa, and the brain region is enlarged to the size of the medical image IMGp1 after pre-processing. In this case, the brain region is maintained and enhanced in the medical image IMGp1 after the image matting step S121, such that critical information for training is extracted and kept in the medical image IMGp1 after pre-processing.

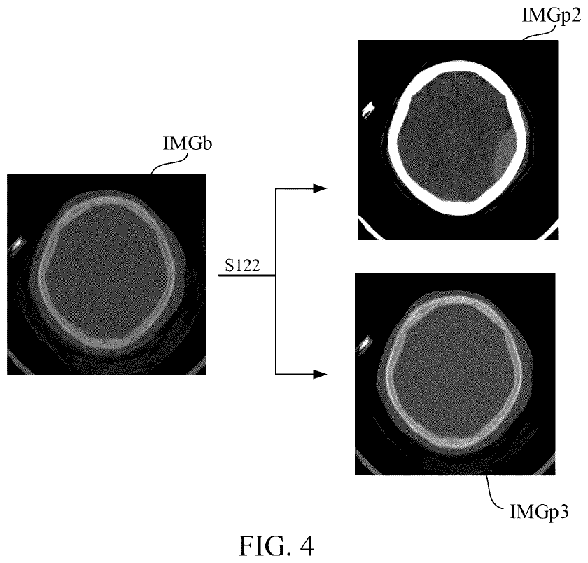

Reference is further made to FIG. 4, which is a schematic diagram illustrating an image windowing step S122 performed on an original medical image IMGb to generate medical images IMGp2 and IMGp3 after pre-processing. As shown in FIG. 4, during the image windowing step S122, pixel values of the original medical image IMGb are adjusted for contrast mapping. In this case, a subdural windowing is performed to the original medical image IMGb to generate the medical image IMGp2 after pre-processing, and a bone windowing is performed to the original medical image IMGb to generate another medical image IMGp3 after pre-processing. In this case, features of subdural or bone are more visible in the medical images IMGp2 and IMGp3 after pre-processing.

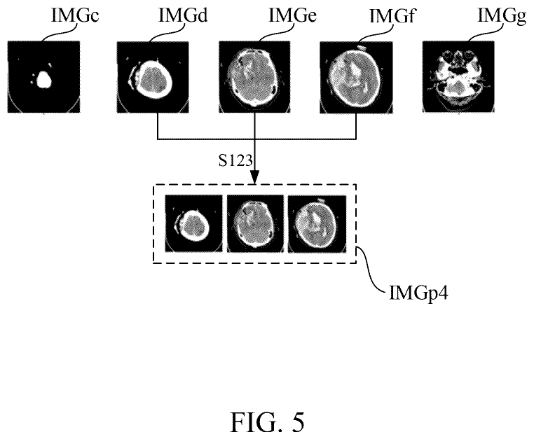

Reference is further made to FIG. 5, which is a schematic diagram illustrating an image stacking step S123 performed on a sequence of original medical images IMGc, IMGd, IMGe, IMGf and IMGg to generate a medical image IMGp4 after pre-processing. As shown in FIG. 5, the original medical images IMGc, IMGd, IMGe, IMGf and IMGg can be sequential images captured in one head computed tomography examination to a patient. During the sequential image stacking step S123, the original medical images in adjacent sequential order are integrated/stacked into the medical images after pre-processing. For example, the IMGd, IMGe and IMGf can be stacked into one medical image IMGp4 after pre-processing. Similarly, other pairs of adjacent medical images can also be stacked into the medical images after pre-processing. In this case, sequential features in adjacent scan images are stored/integrated in the medical image IMGp4 after pre-processing.

In some embodiments, these medical images after pre-processing are utilized in following steps S130-S170 of the multi-label classification method 100 instead of original medical images. In some other embodiments, the step S120 of image-preprocessing can be skipped, and the original medical images can be utilized in following steps 5130-5170 of the multi-label classification method 100.

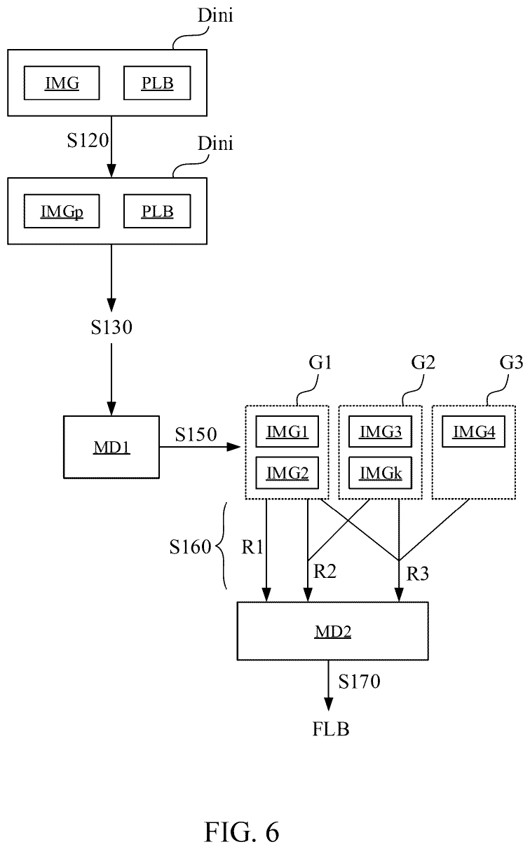

Reference is further made to FIG. 6, which is a schematic diagram illustrating the multi-label classification method 100 in FIG. 1 in some embodiments. As shown in FIG. 1 and FIG. 5, the medical images IMG in the initial dataset Dini are pre-processed in step S120. In this case, the initial dataset Dini includes the medical images IMGp after pre-processing and the partial input labels PLB.

In some embodiments, as shown in FIG. 1, FIG. 2 and FIG. 6, step S130 is executed by the processing unit 240 to train a first multi-label classification model MD1 with the initial dataset Dini, which includes the medical images IMGp after pre-processing and the partial input labels PLB. In some embodiments, the first multi-label classification model MD1 includes a convolutional neural network (CNN). The convolutional neural network may include some convolutional layers, activation layers, pooling layers and/or full-connection layers for classification. The first multi-label classification model MD1 can be trained according to a reward policy with a backpropagation algorithm. The reward policy is defined according to a loss function. In some embodiments, the first multi-label classification model MD1 is trained based on the Masked Binary Cross-Entropy Loss function according to the partial input labels PLB without considering the unlabeled part of the abnormal features.

During step S130, the first multi-label classification model MD1 is executed to generate predictions of the different abnormal features corresponding to the medical images IMGp, and the predictions are compared with the partial input labels PLB to calculate a loss value for adjusting weights/parameters in the convolutional neural network of the first multi-label classification model MD1.

In some embodiments, the processing unit 240 calculates the Masked Binary Cross-Entropy Loss function as:

$$\frac{1}{\sqrt{{\sum }_{i = 1}^{N} {\prod }_{{U}^{′}} ( {y}_{i} )}} {\sum }_{i = 1}^{N} {\prod }_{{U}^{′}} ( {y}_{i} ) ( {y}_{i} log {\hat{y}}_{ι} + ( 1 - {y}_{i} ) log ( 1 - {\hat{y}}_{ι} ) ) \qquad equation ( 1 )$$

$${\prod }_{{U}^{′}} ( {y}_{i} ) = \{ \begin{matrix}1 , & {y}_{i} is not unknown label \\ 0 , & {y}_{i} is unkown label\end{matrix}$$

As shown in equation (1), yᵢ are real labels of the medical images (based on the partial input labels PLB) in the initial dataset Dini, and ŷᵢ are predicted labels generated by the first multi-label classification model MD1. According to the Masked Binary Cross-Entropy Loss function, the loss value will not be affected when yᵢ is an unknown label in the initial dataset Dini.

As shown in FIG. 1, FIG. 2 and FIG. 6, step S140 is executed by the processing unit 240 to estimate difficulty levels of the medical images IMGp in the initial dataset based on predictions generated by the first multi-label classification model MD1.

After the first multi-label classification model MD1 is trained, the first multi-label classification model MD1 is able to generate probability values corresponding to each of the abnormal features on every medical image as shown in Table 3.

TABLE 3

| | IPH | IVH | SAH | SDH | EDH |
|---|---|---|---|---|---|
| IMG1 | 0.82 | 0.22 | 0.15 | 0.2 | 0.34 |
| IMG2 | 0.55 | 0.89 | 0.35 | 0.1 | 0.70 |
| IMG3 | 0.29 | 0.77 | 0.95 | 0.20 | 0.15 |
| IMG4 | 0.15 | 0.45 | 0.66 | 0.73 | 0.35 |
| . . . | . . . | . . . | . . . | . . . | . . . |
| IMGk | 0.33 | 0.20 | 0.56 | 0.37 | 0.77 |

In Table 3, the probability value about the abnormal feature “IPH” on the first medical image IMG1 is “0.82”, which is closer to 1, and it means that the first multi-label classification model MD1 predicts that the first medical image IMG1 is more likely to have the abnormal feature “IPH”. The probability value about the abnormal feature “IVH” on the first medical image IMG1 is “0.22”, which is closer to 0, and it means that the first multi-label classification model MD1 predicts that the first medical image IMG1 is unlikely to have the abnormal feature “IVH”. In some embodiments, the probability values can be generated by the convolutional neural network in the first multi-label classification model MD1.

During the step S140, the processing unit 240 estimates difficulty levels according to a difficulty estimation function as:

L(y, ŷ)=|y−ŷ| equation (2)

In the equation (2), y is a real label about one abnormal feature of the medical images (based on the partial input labels PLB) in the initial dataset Dini. When the label is positive, y=1. When the label is negative, y=O. In the equation (2), ŷ is a corresponding probability values generated by the first multi-label classification model MD1 about the abnormal feature. When a difference between y and ŷ is larger, it means a bigger gap between the prediction made by first multi-label classification model MD1 and the real label, and it also indicates the corresponding image is harder to predict. When a difference between y and ŷ is smaller, it means that the prediction made by first multi-label classification model MD1 is closer to the real label, and it also indicates the corresponding image is easier to predict.

In some embodiments, five difficulty values are estimated in step S140 relative to five abnormal features for each of the medical images IMGp. A maximum among these five difficulty values is regarded as the difficulty level of the target medical image. Reference is further made to Table 4, which is a list of difficulty levels of the medical images IMG1˜IMGk according to the demonstrational example.

TABLE 4

| | difficulty |
|---|---|
| | level |
| IMG1 | 0.16 |
| IMG2 | 0.11 |
| IMG3 | 0.29 |
| IMG4 | 0.35 |
| . . . | . . . |
| IMGk | 0.23 |

As shown in FIG. 1, FIG. 2 and FIG. 6, step S150 is executed by the processing unit 240 to divide the medical images IMG1˜IMGk (after pre-processing) in the initial dataset Dini into different subsets G1-G3 based on difficulty levels of the medical images IMG˜IMGk estimated above.

For example, the medical images IMG1 and IMG2 with the lowest difficulty level can be divided in the first subset G1; the medical images IMG3 and IMGk with the second lowest difficulty level can be divided in the second subset G2; and, the medical image IMG4 with a relatively higher difficulty level can be divided in the third subset G3.

In some embodiments, the medical images IMG1-IMGk can be divided into 10 different subsets based on difficulty levels. The disclosure is not limited to a specific amount of subsets, and the amount of subsets can be adjusted according to practical applications and data characteristic.

As shown in FIG. 1 and FIG. 2, step S160 is executed by the processing unit 240 to train a second multi-label classification model based on subsets G1˜G3 with gradually increasing difficulty levels during different curriculum learning rounds.

As the demonstrational example shown in FIG. 6, during a first curriculum learning round R1 of step S160, the second multi-label classification model MD2 is trained at first based on the first subset G1 (e.g., the medical images IMG1 and IMG2, and the corresponding partial input labels PLB thereof) with the lowest difficulty level. In some embodiments, the first curriculum learning round R1 can be configured to have a time length of one epoch computation time.

Afterward, during a second curriculum learning round R2 of step S160, the second multi-label classification model MD2 is trained again based on the first subset G1 and also the second subset G2 (e.g., the medical images IMG3 and IMGk, and the corresponding partial input labels PLB thereof). The second subset G2 has a difficulty level higher than the first subset G1. In some embodiments, the second curriculum learning round R2 can be configured to have a time length of one epoch computation time.

Afterward, during a third curriculum learning round R3 of step S160, the second multi-label classification model MD2 is trained again based on the first subset G1, the second subset G2 and also the third subset G3 (e.g., the medical image IMG4 and the corresponding partial input label PLB thereof). The third subset G3 has a difficulty level higher than the first subset G1 and the second subset G2. In some embodiments, the third curriculum learning round R3 can be configured to have a time length of one epoch computation time.

As shown in aforesaid embodiments, the second multi-label classification model MD2 is firstly trained with the first subset G1 with the lowest difficulty level. The second multi-label classification model MD2 can establish prediction accuracy based on the easier training data. Afterward, the second multi-label classification model MD2 is repeatedly trained based on increasing subsets with increasing difficulty levels during different curriculum learning rounds. In this case, the second multi-label classification model MD2 can establish capability to handle the harder training data in each curriculum learning rounds.

There curriculum learning rounds R1 to R3 shown in FIG. 6 are illustrated for demonstrational purposes. The disclosure is not limited thereto. In some embodiments, if the medical images in the initial dataset Dini are divided into two subsets, there will be two curriculum learning rounds for sequentially training the second multi-label classification model MD2. In other embodiments, if the medical images in the initial dataset Dini are divided into ten subsets, there will be ten curriculum learning rounds for sequentially training the second multi-label classification model MD2.

In some embodiments, the second multi-label classification model MD2 includes a convolutional neural network (CNN). The convolutional neural network may include some convolutional layers, activation layers, pooling layers and/or full-connection layers for classification. The second multi-label classification model MD2 can be trained according to a reward policy with a backpropagation algorithm. The reward policy is defined according to a loss function. In some embodiments, the second multi-label classification model MD2 is trained based on the Masked Binary Cross-Entropy Loss function according to the subsets selected in each curriculum learning round and the corresponding partial input labels PLB.

As shown in FIG. 1 and FIG. 2, step S170 is executed by the processing unit 240 to generate predicted labels FLB annotated on the medical images about each of the abnormal features, based on the second multi-label classification model MD2.

In the demonstrational example, the second multi-label classification model MD2 is capable to generate five predicted labels about positive or negative predictions of IPH, IVH, SAH, SDH and EDH corresponding to one medical image, as shown in Table 5, which is a list of predicted labels FLB annotated on the medical images about each of the abnormal features according to a demonstrational example.

TABLE 5

| | IPH | IVH | SAH | SDH | EDH |
|---|---|---|---|---|---|
| IMG1 | positive | negative | negative | negative | negative |
| IMG2 | positive | positive | negative | negative | positive |
| IMG3 | negative | positive | positive | negative | negative |
| IMG4 | negative | negative | positive | positive | negative |
| . . . | . . . | . . . | . . . | . . . | . . . |
| IMGk | negative | negative | positive | negative | positive |

As shown in Table 5, the predicted labels FLB generated by the second multi-label classification model MD2 are fully annotated about all of the abnormal features IPH, IVH, SAH, SDH and EDH on every medical image. In this case, there will no missing/unknown label existed in the predicted labels FLB.

The predicted labels FLB generated by the second multi-label classification model MD2 can be utilized for providing diagnosis guides to doctors or healthcare providers and also providing effective treatments to patients. As shown in FIG. 2, the predicted labels FLB (as listed in Table 5) can be displayed on the displayer 280. In some cases, the healthcare providers (or the patients) can quickly acknowledge predicted results about all abnormal features IPH, IVH, SAH, SDH and EDH on patient's medical images captured in the head computed tomography (CT) images, such that the healthcare providers can quickly and accurately react (e.g., providing treatments or medical suggestions) based on the detected abnormal features.

There are possibilities that the second multi-label classification model MD2 may generate wrong predictions among the predicted labels FLB. In some embodiments, the predicted labels FLB are desired to be reviewed by a doctor, a medical laboratory scientist or a clinical scientist.

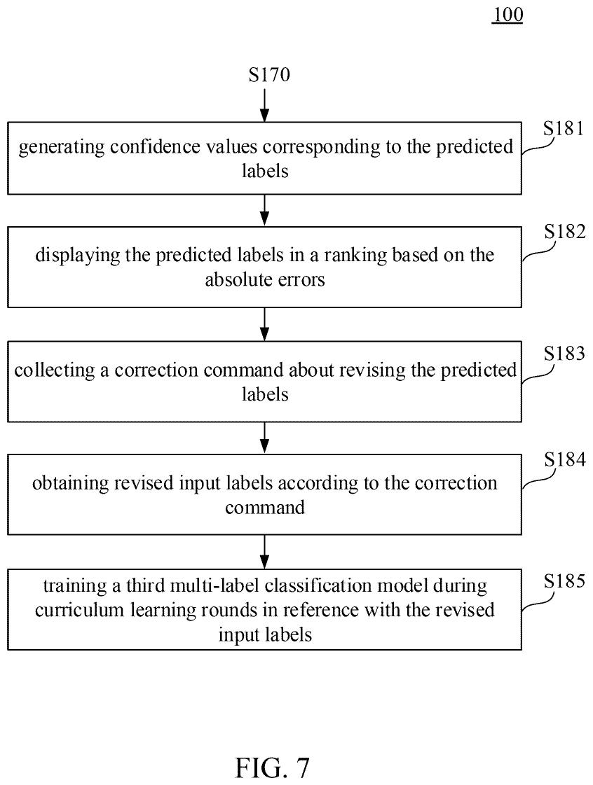

Reference is made to FIG. 7, which is a flow chart diagram illustrating further steps of the multi-label classification method 100 in FIG. 1 according to some embodiments of the disclosure. As shown in FIG. 7, after the multi-label classification method 100 finishes steps S110 to S170 discussed in aforesaid embodiments, the multi-label classification method 100 further includes steps S181 to S185 about reviewing and revising the predicted labels.

As shown in FIG. 2 and FIG. 7, the processing unit 240 is configured to perform step S181 to generate confidence values corresponding to the predicted labels FLB based on the second multi-label classification model MD2.

The confidence values are about affirmative degrees of the second multi-label classification model MD2 about the predicted labels FLB. In some embodiments, the confidence values can be generated by the convolutional neural network in the second multi-label classification model MD2. If the second multi-label classification model MD2 is more affirmative about a target predicted label, the confidence value about the target predicted label will be closer to 1. If the second multi-label classification model MD2 is less affirmative about the target predicted label, the confidence value about the target predicted label will be closer to 0. For example, if the confidence value about the abnormal feature “IPH” is “0.82”, which is closer to 1, and it means that the second multi-label classification model MD2 predicts that the medical image is more likely to have the abnormal feature “IPH”. The confidence value about the abnormal feature “IVH” on the medical image is “0.22”, which is closer to 0, and it means that the second multi-label classification model MD2 predicts that the medical image is unlikely to have the abnormal feature “IVH”.

In this case, the processing unit 240 is configured to generate confidence values for each of the predicted labels FLB. The processing unit 240 is configured to compare the predicted labels FLB generated by the second multi-label classification model MD2 with the input labels (based on the partial input labels PLB in the initial dataset Dini). It is possible that some of the predicted labels FLB can be different from the input labels. When the predicted labels FLB mismatch with the input labels, in step S182, mismatched predicted labels FLB can be displayed on the displayer 280 in a ranking based on an absolute error calculated based on the confidence values and the partial input labels PLB. In some embodiments, the absolute error can be calculated based on the confidence values and the partial input labels PLB, in a way similar to the difficulty estimation function shown in equation (2). For example, the absolute error is calculated by a following equation (3):

absolute error=|y−ĉ| equation (3)

As shown in equation (3), y is a real label about one abnormal feature of the medical images (based on the partial input labels PLB) in the initial dataset Dini. When the label is positive, y=1. When the label is negative, y=0. In the equation (3), ĉ is a corresponding confidence values generated by the second multi-label classification model MD2 about the abnormal feature. When a difference between y and ĉ is larger, it means a bigger gap between the prediction made by second multi-label classification model MD2 and the real label. When a difference between y and is smaller, it means that the prediction made by second multi-label classification model MD2 is closer to the real label.

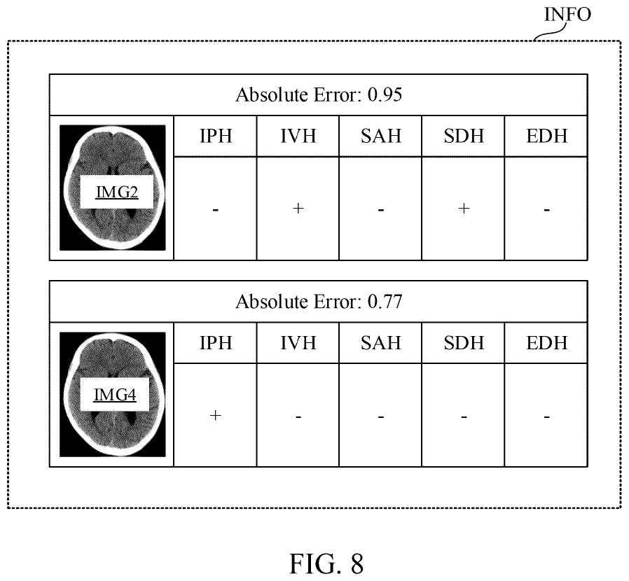

Reference is further made to FIG. 8, which is a schematic diagram illustrating information INFO about mismatched predicted labels on the displayer 280 according to some embodiments.

It is assumed that the predicted labels FLB of the second medical image IMG2 are different from the partial input labels PLB, and the absolute error of the second medical image IMG2 is calculated to be “0.95”. It means that the predicted labels FLB generated by the second multi-label classification model MD2 about the second medical image IMG2 differs from the initial labelling in the partial input labels PLB, and at the same time the second multi-label classification model MD2 has a high confidence level in its prediction. There are two potential causes to this situation. The partial input labels PLB may include errors regarding to the second medical image IMG2, or the predicted labels FLB are wrong predictions. In this case, it requires more attentions from medical personnel to review the labels annotated on the second medical image IMG2. As shown in FIG. 8, the displayed information INFO includes predicted labels FLB about the second medical image IMG2 displayed at a top position with a first ranking.

It is assumed that the predicted labels FLB of the fourth medical image IMG4 are different from the partial input labels PLB, and the absolute error of the fourth medical image IMG4 is calculated to be “0.77”. In this case, it also requires attentions from medical personnel to review labels annotated on the fourth medical image IMG4. As shown in FIG. 8, the displayed information INFO includes predicted labels FLB about the fourth medical image IMG4 displayed with a second ranking below the second medical image IMG2.

Similarly, if there are more medical images with predicted labels different from the partial input labels PLB, these medical images can be displayed on the displayer 280 in a ranking according to their absolute error.

In this case, the medical personnel (e.g., doctor, medical laboratory scientist or clinical scientist) can effectively review the mismatched predicted labels in the ranking of absolute error. If the medical personnel finds out an error within the predicted labels FLB, the medical personnel is able to input a correction command, which may include agreement or disagreement in view of the medical personnel about the predicted labels FLB, through the input interface 220.

As shown in FIG. 2 and FIG. 7, in step S183, the input interface 220 is configured to collect a correction command CMD about revising the predicted labels FLB. The correction command CMD can include an agreement or disagreement about the predicted labels FLB.

In step S184, the processing unit 240 is able to obtain revised input labels according to the correction command CMD. The revised input labels can be generated by integrating labels from the partial input labels PLB and the correction command CMD. In some embodiments, the correction command CMD (collected based on manually inputs from the medical personnel) has a higher priority than the partial input labels PLB in the integration.

In step S185, the processing unit 240 is configured to train a third multi-label classification model during curriculum learning rounds in reference with the revised input labels. In this case, since the revised input labels are reviewed and verified by the medical personnel, the revised input labels have a higher creditability than the partial input labels PLB, which can be automatically generated based on medical records, such that the third multi-label classification model during curriculum learning rounds in reference with the revised input labels can achieve a higher accuracy than the second multi-label classification model MD2. Details about training the third multi-label classification model during curriculum learning rounds in reference with the revised input labels in step S185 are similar to step S160 for training the second multi-label classification model MD2 in reference with the partial input labels PLB, and not to be repeated here again.

Based on aforesaid embodiments, a little effort is required from the medical personnel to verify and review mismatched labels generated by the second multi-label classification model MD2, and the multi-label classification method 100 can automatically produce the revised input labels and train the third multi-label classification model to achieve a higher accuracy. In this case, the multi-label classification model generated by the multi-label classification method 100 can achieve an optimized accuracy in a cost effective manner.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A multi-label classification method, comprising:
obtaining an initial dataset comprising medical images and partial input labels, the partial input labels annotating a labeled part of abnormal features on the medical images;
training a first multi-label classification model with the initial dataset;
estimating difficulty levels of the medical images in the initial dataset based on predictions generated by the first multi-label classification model;
dividing the initial dataset based on the difficulty levels of the medical images into at least a first subset and a second subset, wherein the second subset is estimated to have a higher difficulty level compared to the first subset;
training a second multi-label classification model with the first subset during a first curriculum learning round;
training the second multi-label classification model with the first subset and the second subset during a second curriculum learning round; and
generating, based on the second multi-label classification model, predicted labels annotated on the medical images about each of the abnormal features.

2. The multi-label classification method of claim 1, wherein before training the first multi-label classification model, the multi-label classification method further comprises:
performing an image pre-processing to the medical images in the initial dataset.

3. The multi-label classification method of claim 2, wherein the image pre-processing comprises at least one of image matting, image windowing and sequential image stacking.

4. The multi-label classification method of claim 1, wherein each of the medical images is potentially subject to M abnormal features, the partial input labels indicate positive or negative input labels about N abnormal features, M and N are positive integers and M>N, an unlabeled part of the abnormal features is unknown corresponding to the medical images in the initial dataset.

5. The multi-label classification method of claim 4, wherein the first multi-label classification model comprises a convolutional neural network, and the first multi-label classification model is trained based on a Masked Binary Cross-Entropy Loss function according to the partial input labels without considering the unlabeled part of the abnormal features.

6. The multi-label classification method of claim 1, wherein estimating the difficulty levels of the medical images comprises:
generating, by the first multi-label classification model, probability values for each of the abnormal features relative to the medical images; and
estimating the difficulty levels based on a difficulty estimation function according to the probability values and the partial input labels.

7. The multi-label classification method of claim 1, wherein the second multi-label classification model comprises a convolutional neural network and the first multi-label classification model is trained based on a Masked Binary Cross-Entropy Loss function.

8. The multi-label classification method of claim 1, wherein the medical images comprise head computed tomography (CT) images.

9. The multi-label classification method of claim 8, wherein the abnormal features comprise intraparenchymal hemorrhage (IPH), intraventricular hemorrhage (IVH), subarachnoid hemorrhage (SAH), subdural intracranial hemorrhage (SDH) and epidural hemorrhage (EDH).

10. The multi-label classification method of claim 9, wherein the second multi-label classification model is utilized to generate five predicted labels about positive or negative predictions of IPH, IVH, SAH, SDH and EDH corresponding to one medical image.

11. The multi-label classification method of claim 1, further comprising:
generating, by the second multi-label classification model, confidence values corresponding to the predicted labels;
calculating an absolute error based on the confidence values and the partial input labels; and
displaying the predicted labels in a ranking based on the absolute error.

12. The multi-label classification method of claim 11, further comprising:
collecting a correction command about revising the predicted labels;
obtaining revised input labels according to the correction command; and
training a third multi-label classification model during curriculum learning rounds in reference with the revised input labels.

13. A multi-label classification system, comprising:
a storage unit, configured to store computer-executable instructions; and
a processing unit, coupled with the storage unit, the processing unit is configured to execute the computer-executable instructions to implement a first multi-label classification model and a second multi-label classification model, the processing unit is configured to:
obtain an initial dataset comprising medical images and partial input labels, the partial input labels annotating a labeled part of abnormal features on the medical images;
train the first multi-label classification model with the initial dataset;
estimate difficulty levels of the medical images in the initial dataset based on predictions generated by the first multi-label classification model;
divide the initial dataset based on the difficulty levels of the medical images into at least a first subset and a second subset, wherein the second subset is estimated to have a higher difficulty level compared to the first subset;
train the second multi-label classification model with the first subset during a first curriculum learning round;
train the second multi-label classification model with the first subset and the second subset during a second curriculum learning round; and
utilize the second multi-label classification model to generate predicted labels annotated on the medical images about each of the abnormal features.

14. The multi-label classification system of claim 13, wherein before training the first multi-label classification model, the processing unit is further configured to perform an image pre-processing to the medical images in the initial dataset, the image pre-processing comprises at least one of image matting, image windowing and sequential image stacking.

15. The multi-label classification system of claim 13, wherein each of the medical images is potentially subject to M abnormal features, the partial input labels indicate positive or negative input labels about N abnormal features, M and N are positive integers and M>N, an unlabeled part of the abnormal features is unknown corresponding to the medical images in the initial dataset.

16. The multi-label classification system of claim 15, wherein the first multi-label classification model comprises a convolutional neural network, and the first multi-label classification model is trained based on a Masked Binary Cross-Entropy Loss function according to the partial input labels without considering the unlabeled part of the abnormal features.

17. The multi-label classification system of claim 13, wherein the processing unit estimates the difficulty levels of the medical images by:
generating, by the first multi-label classification model, probability values for each of the abnormal features relative to the medical images; and
estimating the difficulty levels based on a difficulty estimation function according to the probability values and the partial input labels.

18. The multi-label classification system of claim 13, wherein the medical images comprise head computed tomography (CT) images, and the abnormal features comprise intraparenchymal hemorrhage (IPH), intraventricular hemorrhage (IVH), subarachnoid hemorrhage (SAH), subdural intracranial hemorrhage (SDH) and epidural hemorrhage (EDH), and the second multi-label classification model is utilized to generate five predicted labels about positive or negative predictions of IPH, IVH, SAH, SDH and EDH corresponding to one medical image.

19. The multi-label classification system of claim 13, further comprising:
a displayer, coupled with the processing unit, wherein the processing unit is configured to generate confidence values corresponding to the predicted labels based the second multi-label classification model, the processing unit is configured to calculate an absolute error based on the confidence values and the partial input labels, the displayer is configured to display the predicted labels in a ranking based on the absolute error.

20. The multi-label classification system of claim 19, further comprising:
an input interface, coupled with the processing unit, wherein the input interface is configured to collect a correction command about revising the predicted labels, the processing unit is configured to obtain revised input labels according to the correction command and train a third multi-label classification model during curriculum learning rounds in reference with the revised input labels.
